(19) United States
(12) Patent Application Publication
(10) Pub. No.: US 2021/0259659 A1
(43) Pub. Date: Aug. 26, 2021

(54) INTRAVASCULAR ULTRASOUND ROTATION TRACKING

(71) Applicant: KONINKLIJKE PHILIPS N.V., EINDHOVEN (NL)

(72) Inventors: Frank Michael WEBER, HAMBURG (DE); Michael GRASS, BUCHHOLZ IN DER NORDHEIDE (DE)

(21) Appl. No.: 17/250,150

(22) PCT Filed: Jun. 18, 2019
(86) PCT No.: PCT/EP2019/066071

§ 371 Date: Dec. 7, 2020

Related U.S. Application Data
(60) Provisional application No. 62/687,053, filed on Jun. 19, 2018.

(51) Int. Cl. A61B 8/08; A61B 8/12; A61B 8/00

(52) U.S. Cl. CPC A61B 8/0841; A61B 8/4245; A61B 8/483; A61B 8/12

(57) ABSTRACT

A controller (260) for identifying rotation of an interventional medical device (252) includes a memory that stores instructions and a processor that executes the instructions. When executed by the processor, the instructions cause the controller (260) to execute a process that includes receiving (S410) a first signal emitted from the interventional medical device (252) and corresponding to a first predetermined direction relative to the interventional interventional medical device (252) relative to a fixed rotation.

### BACKGROUND

Intravascular ultrasound imaging probes are used to image blood vessels such as coronary arteries to analyze plaque in the vessels or the positioning and the expansion of stents. The intravascular ultrasound probe is provided as/with a catheter inserted into the vessel and typically includes one sensor ring or a rotating sensor that acquires images looking radially outwards in all directions, thus imaging one cross-section of the vessel essentially in 2-dimensions. To image the complete vessel the intravascular ultrasound probe is pulled back, and the series of 2-dimensional cross-sections is stacked to generate 3-dimensional imagery such as a 3-dimensional ultrasound volume.

Intravascular ultrasound rotation tracking as described herein relates to detecting a rotational degree of freedom of interventional medical devices such as the intravascular ultrasound imaging probes. Knowledge of the rotation can be important both for reconstructing intravascular ultrasound imagery and for fusing the intravascular ultrasound imagery with additional imagery such as transesophageal echocardiogram (TEE) imagery or transthoracic echocardiogram (TTE) imagery.

One use for intravascular ultrasound imaging probes is for interventional cardiac procedures. Interventional cardiac procedures may be guided by any of a multitude of different types of imaging devices, including X-ray machines and ultrasound imaging probes. Besides intravascular ultrasound, examples of ultrasound used in interventional cardiac procedures include transesophageal echocardiogram, transthoracic echocardiogram and intracardiac echocardiogram (ICE). Since the ultrasound imaging probes are moveable, accurate registration relative to a fixed patient coordinate system or relative to the imaging coordinates of other imaging devices is an issue. Registration involves setting different systems such as ultrasound imaging probes and fixed patient coordinate systems to a common coordinate system with a common origin, so that imagery from the different systems is visible in an accurate and comparable context.

Intravascular ultrasound imaging poses two problems with respect to rotation. First, the intravascular ultrasound imaging probe may be rotated during the pullback, but the rotation is not detected and therefore not considered when combining the 2-dimensional cross-sections. Second, the overall orientation of the intravascular ultrasound imaging probe (and thus the intravascular ultrasound imagery) with respect to the patient anatomy is not known. If a transesophageal echocardiogram ultrasound imaging probe is used to image the anatomy, it is unknown, for example, which part of the intravascular ultrasound imagery is acquired in the direction facing the transesophageal echocardiogram ultrasound imaging probe. This makes it impossible to fuse the intravascular ultrasound imagery with the transesophageal echocardiogram imagery (or an anatomical model created from the transesophageal echocardiogram imagery).

To address the uncertainty that exists as to rotation and orientation, intravascular ultrasound rotation tracking has been developed.

### SUMMARY

According to an aspect of the present disclosure, a controller for identifying rotation of an interventional medical device includes a memory that stores instructions and a processor that executes the instructions. When executed by the processor, the instructions cause the controller to execute a process that includes receiving a first signal emitted from the interventional medical device and corresponding to a first predetermined direction relative to the interventional medical device, and determining, based on the first signal, a first rotation of the interventional medical device relative to a fixed rotation.

According to another aspect of the present disclosure, a method for identifying rotation of an interventional medical device includes receiving a first signal emitted from the interventional medical device and corresponding to a first predetermined direction relative to the interventional medical device, and determining, based on the first signal, a first rotation of the interventional medical device relative to a fixed rotation.

According to still another aspect of the present disclosure, a system for identifying rotation of an interventional medical device includes the interventional medical device, an ultrasound imaging probe, and a controller with a memory that stores instructions and a processor that executes the instructions. The ultrasound imaging probe captures imagery in a space that includes the interventional medical device. When executed by the processor, the instructions cause the controller to execute a process that includes receiving a first signal emitted from the interventional medical device and corresponding to a first predetermined direction relative to the interventional medical device, and determining, based on the first signal, a first rotation of the interventional medical device relative to a fixed rotation.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates an interventional setup for intravascular ultrasound rotation tracking, in accordance with a representative embodiment.

FIG. 1B illustrates differentiated frequencies in a field of 360 degrees for intravascular ultrasound rotation tracking, in accordance with a representative embodiment.

FIG. 2 illustrates a system for intravascular ultrasound rotation tracking, in accordance with a representative embodiment.

FIG. 3 is an illustrative embodiment of a general computer system, on which a method of intravascular ultrasound rotation tracking can be implemented, in accordance with a representative embodiment.

FIG. 4 illustrates a method for intravascular ultrasound rotation tracking, in accordance with a representative embodiment.

FIG. 5 illustrates another method for intravascular ultrasound rotation tracking, in accordance with a representative embodiment.

FIG. 6 illustrates another method for intravascular ultrasound rotation tracking, in accordance with a representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

In intravascular ultrasound imaging, the intravascular ultrasound imaging probe is inserted in a vessel and delivers a radial image of the vessel. The rotational orientation of the intravascular ultrasound imaging probe inside the vessel is an unknown. In intravascular ultrasound rotation identification described herein, accurate determination of the rotational orientation of the intravascular ultrasound imaging probe relative to an external ultrasound imaging probe used during the medical intervention is performed in order to improve vascular pullback images or for improved registration of the ultrasound imagery from the intravascular ultrasound imaging probe to the ultrasound imagery from the external ultrasound imaging probe.

As described below, rotation and orientation can be determined using a second ultrasound imaging probe such as an external transesophageal echocardiogram or transthoracic echocardiogram probe.

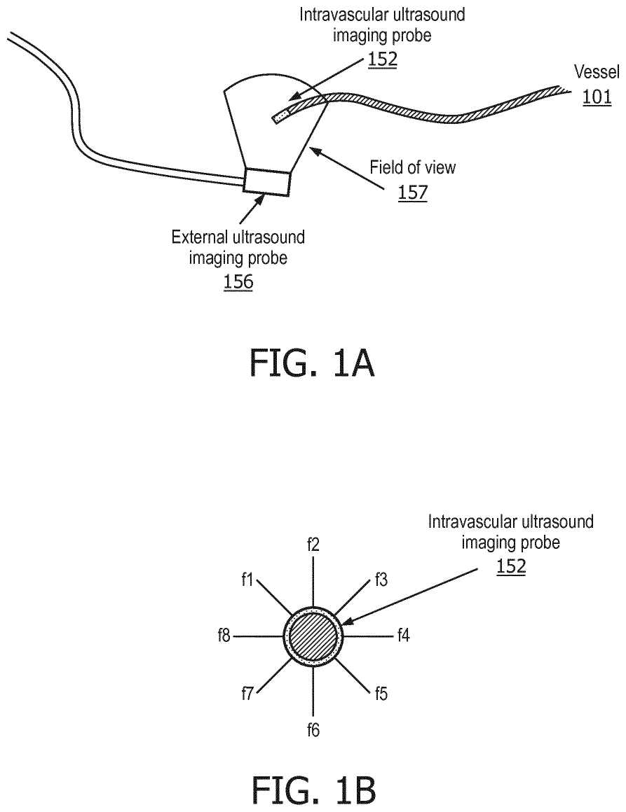

FIG. 1A illustrates an interventional setup for intravascular ultrasound rotation tracking, in accordance with a representative embodiment.

In FIG. 1A, an interventional setup includes an intravascular ultrasound imaging probe (IVUS) 152 that is inserted into a vessel 101, such as by/with a catheter. Another example of an intravascular ultrasound imaging probe 152 is an intracardiac echo (ICE) probe. The intravascular ultrasound imaging probe 152 may be within a field of view 157 of an external ultrasound imaging probe 156, but does not have to be. The field of view 157 may be a region from which an image is acquired. The image is composed of scan lines, and the field of view 157 is a result of the distribution and depth of the scan lines. Images can be acquired with a narrower/shallower field of view 157 to, for example, obtain higher frame rates. When the ultrasound image from the intravascular ultrasound imaging probe 152 is to be fused with the image from the external ultrasound imaging probe 156, the intravascular ultrasound imaging probe 152 should be within the field of view 157. However, for the purposes of rotation tracking, the external ultrasound imaging probe 156 does not necessarily have to acquire images while the intravascular ultrasound imaging probe 152 is pulled back, as the external ultrasound imaging probe 156 may only be detecting signals from the catheter. An example of the external ultrasound imaging probe 156 is a 3-dimensional transesophageal echocardiogram probe, a transthoracic echocardiogram probe, or an intracardiac echo (ICE) probe. The intravascular ultrasound imaging probe 152 is tracked according to intravascular ultrasound rotation tracking described herein.

FIG. 1B illustrates differentiated frequencies in a field of 360 degrees for intravascular ultrasound rotation tracking, in accordance with a representative embodiment.

Intravascular ultrasound rotation tracking may be based in part on frequency differentiation. In FIG. 1B, the intravascular ultrasound imaging probe 152 sends echo signals with different frequencies along various angular directions. Specifically, f1, f2, f3, f4, f5, f6, f7 and f8 represent different frequencies for beacon signals emitted in different directions around a circumference. Each frequency in FIG. 1B corresponds to a different emitting element.

Modern intravascular ultrasound imaging probes use, for example, CMUT (capacitive micromachined ultrasonic transducer) technology such as CMUT elements. The frequency of transmission for CMUT elements can easily be tuned in-place over a large frequency range to differentiate different CMUT elements with different frequencies as shown in FIG. 1B. The CMUT elements are used in two ways. First, the CMUT elements acquire the intravascular ultrasound imagery in a frequency range of 10-20 MHz. Second, the CMUT elements intermittently emit beacon-like signals in the frequency range of the external ultrasound imaging probe 156 around 1-5 MHz. The beacon-like signals are recorded by the external ultrasound imaging probe 156 to track the orientation of the intravascular ultrasound imaging probe 152. Because the available CMUT elements provide the dual use, no additional elements are needed, although tracking could also be performed with additional dedicated CMUT elements. While CMUT elements are primarily described for embodiments herein, they are only used as a convenient example of elements with functionalities attributed to the CMUT elements described herein. Thus, CMUT elements are representative of elements used to emit beacon-like signals and/or acquire intravascular ultrasound imagery. In embodiments, these characteristic functions may be separately performed by different elements, or by alternatives to CMUT elements that still possess the above-noted functionalities.

In FIG. 1B, eight frequencies are shown, but the number of frequencies can be more or less than eight. Additionally, in FIG. 1B, the beacons signals are shown to be emitted in a full circumference, but part of the circumference such as a bottom third may not be provided with CMUT elements in an embodiment, such as when the corresponding intravascular ultrasound imaging probe 152 is not likely to be fully or mostly flipped (i.e., such that the bottom would become the top). Moreover, the beacon signals in FIG. 1B are shown to be dispersed uniformly, but this is not particularly required, and beacon signals may be more concentrated at the top than away from the top. In an embodiment, frequencies may be aligned in order so that close frequencies are used for beacon signals emitted from adjacent or close CMUT elements. Additionally, when CMUT elements are aligned evenly, the programmed angles for the CMUT elements may also be spaced evenly.

Assuming that all beacon signals are powered at the same level, beacon signals emitted towards the external ultrasound imaging probe 156 (typically up) will have a stronger received power on the power-spectrum than other beacon signals. Thus, when analyzing the power-spectrum of received beacon signals at the external ultrasound imaging probe 156, the frequency of the beacon signal with the strongest detected power on the power-spectrum can be used to identify the relative orientation of the corresponding CMUT element on the intravascular ultrasound imaging probe 152. That is, the strongest received power level will correspond to the CMUT element closest to the external ultrasound imaging probe 156, and the strongest received power level can be used to identify the orientation of the intravascular ultrasound imaging probe 152 based on this concept. In other words, since the different elements of the intravascular ultrasound imaging probe 152 can be arranged in a predetermined pattern, identification of the relatively strongest signal received at the external ultrasound imaging probe 156 can be used to identify the relative orientation of the intravascular ultrasound imaging probe.

In an embodiment, the two measurements of the two strongest signals can be interpolated, based on the understanding that a CMUT element will almost never be perfectly aligned with the external ultrasound imaging probe. As an example, a Gaussian curve may be fit to the measured power-spectrum, to determine the peak frequency and in turn the orientation of the intravascular ultrasound imaging probe 152. In an embodiment, a table or map may be used to plot relative power measurements of two beacon signals against a pre-identified orientation. For example, a reading of 90% of one signal (e.g., F7) and 15% of another (e.g., F6) may correspond to a rotation of 17.5.

When the intravascular ultrasound imaging probe 152 and the external ultrasound imaging probe 156 are used in a time correlated setup based on the same clock, a run length of the beacon signals from the CMUT elements in/on the intravascular ultrasound imaging probe 152 can also be analyzed. This analysis enables tracking of the intravascular ultrasound imaging probe 152 relative to the external ultrasound imaging probe 156. In addition, if the distance between the intravascular ultrasound imaging probe 152 and the external ultrasound imaging probe 156 is known, a 3-dimensional registration with respect to a simultaneously-acquired X-ray image is feasible, since the known distance and the 2-dimensional position of the intravascular ultrasound imaging probe 152 and the external ultrasound imaging probe 156 in the X-ray image will allow for accurate depth estimation relative to the X-ray system.

Based on correctly identifying the orientation of the intravascular ultrasound imaging probe 152, intravascular ultrasound image reconstruction can be improved. Additionally, the correct identification allows or enhances the ability to combine intravascular ultrasound imagery with transesophageal echocardiogram imagery and transthoracic echocardiogram imagery.

Incidentally, it should be noted that movement of a catheter that includes the intravascular ultrasound imaging probe 152 should have only a negligible effect on frequencies used in intravascular ultrasound rotation tracking. In particular, movement of a catheter can theoretically change the recorded frequency due to the Doppler effect. However, because ultrasound velocity is in the order of c=1000 m/s, and catheter movement in the order v=1 cm/s. Relative frequency shift should be at most v/c, so that would be 0.001%. Thus, in practice, the effect is negligible.

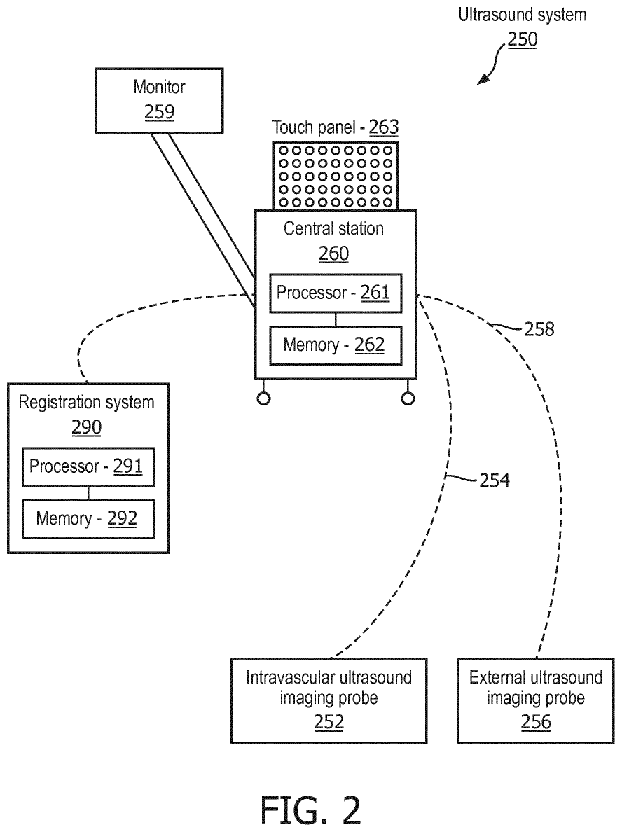

FIG. 2 illustrates a system for intravascular ultrasound rotation tracking, in accordance with a representative embodiment.

In FIG. 2, an ultrasound system 250 includes a central station 260 with a processor 261 and memory 262, a touch panel 263, a monitor 259, an external ultrasound imaging probe 256 connected to the central station 260 by a data connection 258 (e.g., a wired or wireless data connection), and an intravascular ultrasound imaging probe 252 connected to the central station 260 by a data connection 254 (e.g., a wired or wireless data connection). Although not shown, the external ultrasound imaging probe 256 and the intravascular ultrasound imaging probe 252 may each have their own station such as central station 260. In other embodiments, data integration that is performed by the central station 260 in FIG. 2 may be performed in the cloud (i.e., by distributed computers such as at data centers), or by a station dedicated to one or the other of the external ultrasound imaging probe 256 or the intravascular ultrasound imaging probe. Thus, the configuration shown in FIG. 2 is representative of a variety of configurations that would perform image processing and related functionality as described herein.

A registration system 290 includes a processor 291 and a memory 292, and registers ultrasound imagery from the intravascular ultrasound imaging probe 252 to ultrasound imagery from the external ultrasound imaging probe 256. In other words, ultrasound imagery from the intravascular ultrasound imaging probe 252 is registered with imagery from the external ultrasound imaging probe 256 by the registration system 290, or another system that performs image registration. The registration system 290 performs processes described herein by, for example, the processor 291 executing instructions in the memory 292. However, the registration system 290 may also be implemented in or by the central station 260, or in any other mechanism. The combination of the processor 291 and memory 292, whether in the registration system 290 or in another configuration, may be considered a “controller” as the term is used herein. A “controller” may also be implemented by a combination of at least a processor 261 and memory 262 in the central station 260, or a similar combination of elements in or provided with (e.g., attached to) the intravascular ultrasound imaging probe 252 or the external ultrasound imaging probe 256. As noted previously, the elements used to provide beacon signals may be CMUT elements.

The intravascular ultrasound imaging probe 252 corresponds to the intravascular ultrasound imaging probe 152 from FIG. 1A and FIG. 1B. Specifically, the intravascular ultrasound imaging probe 252 may be inserted into a vessel and emit beacon signals in multiple different directions in a circumference. The intravascular ultrasound imaging probe 252 may be within a field of view of the external ultrasound imaging probe 256 while in the vessel, and rotation of the intravascular ultrasound imaging probe 252 or a catheter provided with the intravascular ultrasound imaging probe 252 may be tracked and identified over time by a sequence of signals. The rotation can be adjusted so as to align ultrasound imagery from the intravascular ultrasound imaging probe 252, so as to correctly align a series of 2-dimensional ultrasound imagery to create 3-dimensional ultrasound imagery. The 3-dimensional ultrasound imagery created from a sequence of 2-dimensional ultrasound imagery may be a 3-dimensional volume. In embodiments, a first image from the intravascular ultrasound imaging probe 252 may be used as a reference, so that subsequent images from the intravascular ultrasound imaging probe are adjusted relative to the first image. The first image and subsequent images may be a series of images used to create the 3-dimensional volume, so that another first image for another 3-dimensional volume may similarly be used as a reference for subsequent images used to create the other 3-dimensional volume. That is, a reference may be generated from a first image or other image each time a new set of images is acquired, such as to construct the 3-dimensional volume.

The registration system 290 provides for registering the 2-dimensional ultrasound imagery and 3-dimensional ultrasound imagery from the intravascular ultrasound imaging probe 252 to ultrasound imagery from the external ultrasound imaging probe 256. The registration provides a common origin and coordinate system for the different imagery, so as to accurately reflect how the different imagery is related. Imagery from the intravascular ultrasound imaging probe 252 can be fused with imagery from the external ultrasound imaging probe 256, based on the common coordinate system.

By way of explanation, the intravascular ultrasound imaging probe 252 is placed internally into a patient during a medical procedure, and this is reflective of the “intervention” in interventional medical procedures described herein. Locations of the intravascular ultrasound imaging probe 252 can be seen both on imagery generated by the external ultrasound imaging probe 256, as well other types of imaging systems such as X-Ray when used. Alignment of imagery from the intravascular ultrasound imaging probe 252 is maintained to the extent possible. As described herein, data from the external ultrasound imaging probe 256 can be used to detect rotation of the intravascular ultrasound imaging probe 252, which can be used and useful in variety of ways. Additionally, either or both of the intravascular ultrasound imaging probe 252 and the external ultrasound imaging probe 256 may be an intracardiac echocardiogram (ICE) probe.

In an example, upon detecting rotation of the intravascular ultrasound imaging probe 252, imagery from the intravascular ultrasound imaging probe 252 may be compensated to maintain alignment over time. Additionally, registration of the imagery from the intravascular ultrasound imaging probe 252 to imagery from the external ultrasound imaging probe 256 will accurately reflect the rotation of the intravascular ultrasound imaging probe 252.

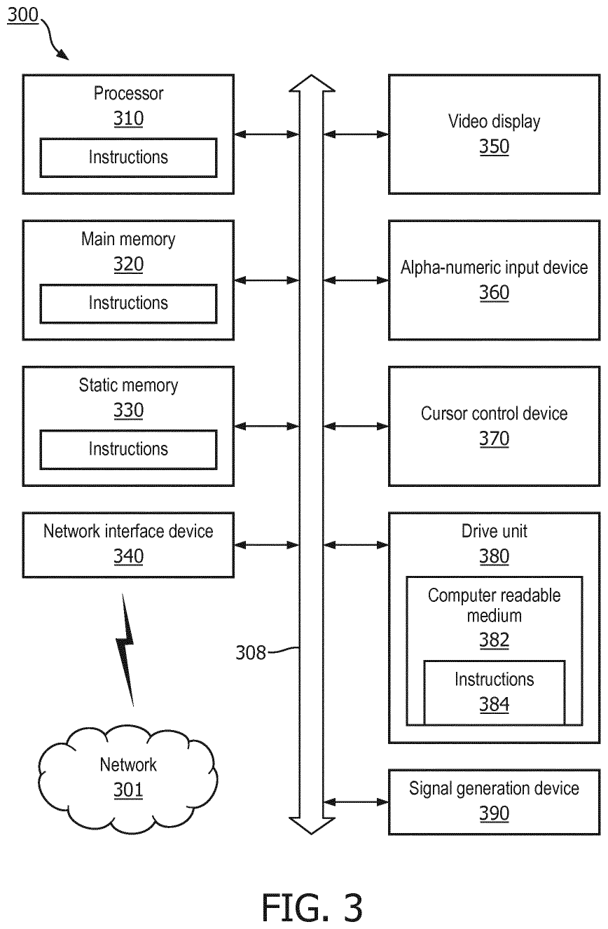

FIG. 3 is an illustrative embodiment of a general computer system, on which a method of intravascular ultrasound rotation tracking can be implemented, in accordance with a representative embodiment. The computer system 300 can include a set of instructions that can be executed to cause the computer system 300 to perform any one or more of the methods or computer based functions disclosed herein. The computer system 300 may operate as a standalone device or may be connected, for example, using a network 301, to other computer systems or peripheral devices.

In a networked deployment, the computer system 300 may operate in the capacity of a server or as a client user computer in a server-client user network environment, or as a peer computer system in a peer-to-peer (or distributed) network environment. The computer system 300 can also be implemented as or incorporated into various devices, such as a stationary computer, a mobile computer, a personal computer (PC), a laptop computer, a tablet computer, a wireless smart phone, an intravascular ultrasound imaging probe, an external ultrasound imaging probe, a central station, a registration system, or any other machine capable of executing a set of instructions (sequential or otherwise) that specify actions to be taken by that machine. The computer system 300 can be incorporated as or in a device that in turn is in an integrated system that includes additional devices. In an embodiment, the computer system 300 can be implemented using electronic devices that provide voice, video or data communication. Further, while a computer system 300 is illustrated in the singular, the term “system” shall also be taken to include any collection of systems or sub-systems that individually or jointly execute a set, or multiple sets, of instructions to perform one or more computer functions.

As illustrated in FIG. 3, the computer system 300 includes a processor 310. A processor for a computer system 300 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A processor is an article of manufacture and/or a machine component. A processor for a computer system 300 is configured to execute software instructions to perform functions as described in the various embodiments herein. A processor for a computer system 300 may be a general-purpose processor or may be part of an application specific integrated circuit (ASIC). A processor for a computer system 300 may also be a microprocessor, a microcomputer, a processor chip, a controller, a microcontroller, a digital signal processor (DSP), a state machine, or a programmable logic device. A processor for a computer system 300 may also be a logical circuit, including a programmable gate array (PGA) such as a field programmable gate array (FPGA), or another type of circuit that includes discrete gate and/or transistor logic. A processor for a computer system 300 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 300 includes a main memory 320 and a static memory 330 that can communicate with each other via a bus 308. Memories described herein are tangible storage mediums that can store data and executable instructions, and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 300 may further include a video display unit 350, such as a liquid crystal display (LCD), an organic light emitting diode (OLED), a flat panel display, a solid-state display, or a cathode ray tube (CRT). Additionally, the computer system 300 may include an input device 360, such as a keyboard/virtual keyboard or touch-sensitive input screen or speech input with speech recognition, and a cursor control device 370, such as a mouse or touch-sensitive input screen or pad. The computer system 300 can also include a disk drive unit 380, a signal generation device 390, such as a speaker or remote control, and a network interface device 340.

In an embodiment, as depicted in FIG. 3, the disk drive unit 380 may include a computer-readable medium 382 in which one or more sets of instructions 384, e.g. software, can be embedded. Sets of instructions 384 can be read from the computer-readable medium 382. Further, the instructions 384, when executed by a processor, can be used to perform one or more of the methods and processes as described herein. In an embodiment, the instructions 384 may reside completely, or at least partially, within the main memory 320, the static memory 330, and/or within the processor 310 during execution by the computer system 300.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 382 that includes instructions 384 or receives and executes instructions 384 responsive to a propagated signal; so that a device connected to a network 301 can communicate voice, video or data over the network 301. Further, the instructions 384 may be transmitted or received over the network 301 via the network interface device 340.

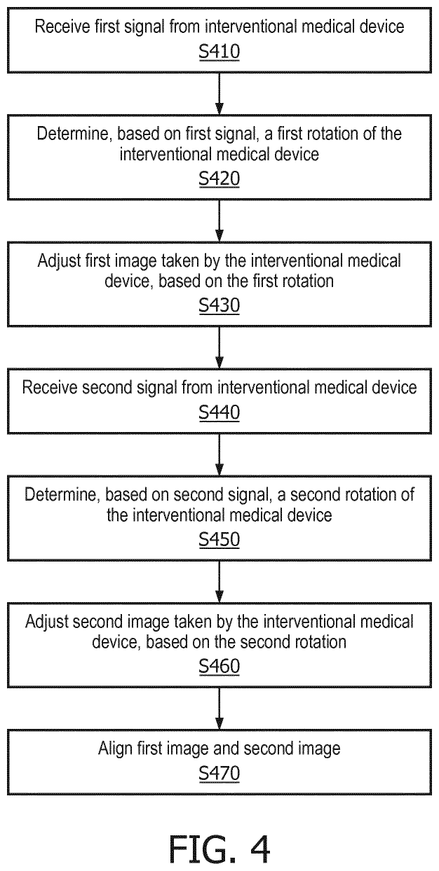

FIG. 4 illustrates a method for intravascular ultrasound rotation tracking, in accordance with a representative embodiment.

In FIG. 4, the method starts at S410 when a first signal is received from an interventional medical device. In the context of intravascular ultrasound rotation tracking, the interventional medical device is representative of a class of medical devices that can be used in an interventional medical procedure and which includes an intravascular ultrasound imaging probe 152 and an intravascular ultrasound imaging probe 252. The first signal is based on one or more beacon signals at predetermined frequencies as in FIG. 1B, and is reflective of information that identifies which of multiple CMUT elements on/in the interventional medical device is closest to an external ultrasound imaging probe 156 or external ultrasound imaging probe 256.

At S420, the method includes determining, based on the first signal, a first rotation of the interventional medical device. Specifically, since the first signal has a measurable power spectrum associated with one or more frequencies, the relative strength of the power at each frequency can be measured. Since the frequencies associated with each beacon signal are known, and the pattern in which the elements are arranged in/on the interventional medical device is known, the relative strength of the power of the first signal at two frequencies reflects the relative distance and/or direction of the corresponding elements from the external ultrasound imaging probe 156 or external ultrasound imaging probe 256. That is, CMUT elements in/on the interventional medical device may emit beacon signals in a predetermined pattern, such that the beacons signals are emitted at predetermined angles relative to a “top” of the interventional medical device. Thus, the beacon signals are emitted at fixed rotational angles, such as relative to the “top” of the interventional medical device, and insofar as the CMUT elements that emit the beacon signals are arranged at the fixed rotational angles from the “top”.

At S430, a first image (ultrasound imagery) taken by the interventional medical device is adjusted based on the first rotation. The adjustment may be simply to align the imagery so that the top of the image corresponds to a predetermined direction (i.e., up).

At S440, a second signal is received from the interventional medical device. Receipt of the second signal at S440 is representative of a sequence of signals that are received, corresponding to a sequence of images that are taken. In other words, the number of signals that may be received may be in the dozens, hundreds, thousands, and even tens of thousands, and can correspond to a like number of images that are sequentially taken.

At S450, a second rotation of the interventional medical device is determined based on the second signal. Specifically, the power spectrum can be analyzed at the different predetermined frequencies in order to determine the relative alignment of CMUT elements in/on the intravascular ultrasound imaging probe 152 or intravascular ultrasound imaging probe 252.

At S460, a second image taken by the interventional medical device is adjusted based on the second rotation. At S470, the first image and the second image are aligned. The alignment of sequential images that may have been rotated by different amounts may be an end use of the intravascular ultrasound rotation tracking described herein.

For the process of FIG. 4 described above, tracking is based on frequency recognition, where analysis of the power spectrum at different frequencies corresponding to a known arrangement of CMUT elements can reflect the relative positions of the CMUT elements.

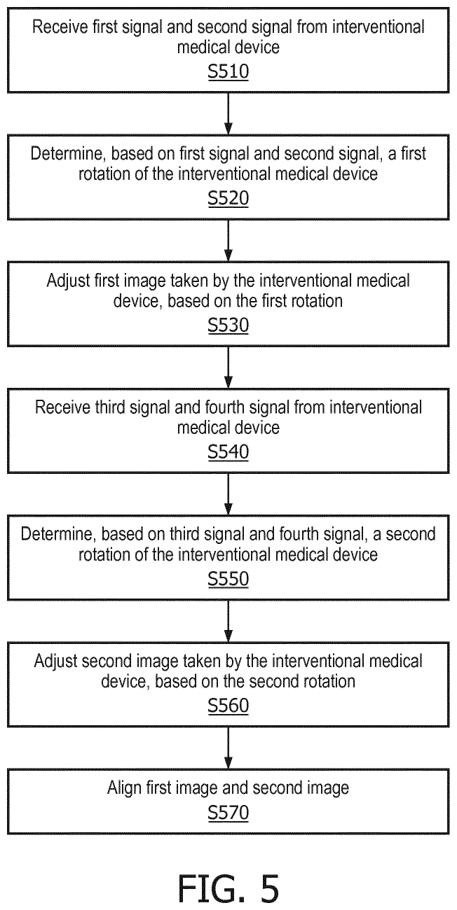

FIG. 5 illustrates another method for intravascular ultrasound rotation tracking, in accordance with a representative embodiment.

The method in FIG. 5 starts at S510 by receiving a first signal and a second signal from an interventional medical device. At S520, a first rotation of the interventional medical device is determined based on the first signal and the second signal. A first image taken by the interventional medical device is adjusted based on the first rotation at S530.

The process in FIG. 5 differs from the process in FIG. 4 in that the first signal and second signal in FIG. 5 may be received simultaneously or near-simultaneously from different elements in/on an intravascular ultrasound imaging probe 152 or intravascular ultrasound imaging probe 252. That is, the first signal and second signal each correspond to a different frequency emitted by a different element, rather than to a combined set of frequencies from multiple elements as for each individual signal in FIG. 4.

At S540, a third signal and a fourth signal are received from the interventional medical device. At S550, a second rotation of the interventional medical device is determined based on the third signal and the fourth signal. At S560, a second image taken by the interventional medical device is adjusted based on the second rotation. At S570, the first image and the second image are aligned.

The first signal and second signal in FIG. 5 are representative of more than one signals at different frequencies emitted for a single image from the intravascular ultrasound imaging probe 152 or intravascular ultrasound imaging probe. The number of signals emitted at different frequencies simultaneously or near-simultaneous is not limited to two (2), and may be twelve (12), thirty-six (36), one hundred (100), or many different numbers. That is, more than two elements may be arranged in/on an intravascular ultrasound imaging probe 152 or intravascular ultrasound imaging probe 252, and each may correspond to a different frequency used in intravascular ultrasound rotation tracking.

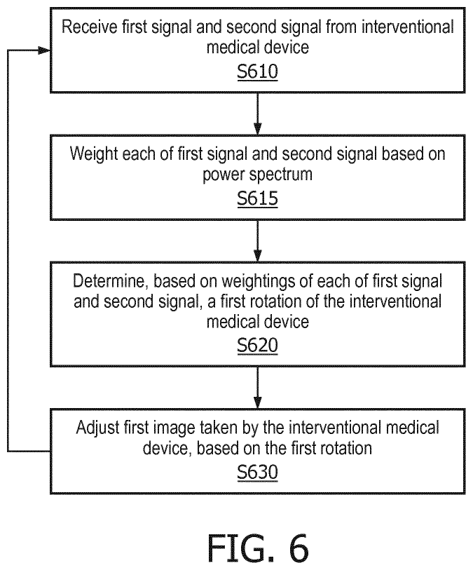

FIG. 6 illustrates another method for intravascular ultrasound rotation tracking, in accordance with a representative embodiment.

In FIG. 6, the method starts at S610 when a first signal and second signal are received from an interventional medical device. At S615, each of the first signal and the second signal is weighted based on the power spectrum corresponding to the first signal and second signal (and any others received at S610). For example, the first signal and the second signal may be linearly weighted based on the strength/intensity of power received at the frequency of the first signal and the (different) frequency of the second signal.

Additionally, the weighting may be performed on only a subset of signals, such as signals that meet a predetermined threshold. In this way, only the signals emitted from elements near the of the intravascular ultrasound imaging probe 152 or intravascular ultrasound imaging probe 252 closest to the external ultrasound imaging probe 156 or external ultrasound imaging probe 256 are weighted, and signals for which only inconsequential trace signals are received may be ignored. As an example, when thirty-six signals are emitted at angles of 10 degrees in a circumference, weighting may be performed on only the five strongest signals, in order to determine the orientation and rotation of the intravascular ultrasound imaging probe 152 or intravascular ultrasound imaging probe 252.

At S620, a first rotation of the interventional medical device is determined based on the weightings of each of the first signal and the second signal. That is, the relative strength/intensity of power received at frequencies of the first signal and the second signal is used to determine the first rotation. For example, a “top” of the interventional medical device determined from the relative signal strength/signal intensity of power received at frequencies of the first signal and the second signal can be compared to a predetermined top direction (i.e., up), and the difference is the rotation of the interventional medical device.

At S630, the first image taken by the interventional medical device is adjusted, based on the first rotation, and the process then returns to S610 for the next image taken by the interventional medical device. As noted previously, a first image may be used as a reference without being adjusted in embodiments, so that subsequent images from the intravascular ultrasound imaging probe are adjusted relative to the first image.

Accordingly, intravascular ultrasound rotation tracking can be used to determine the rotational orientation of an intravascular ultrasound imaging probe using a transesophageal echocardiogram probe, a transthoracic echocardiogram probe, or an intracardiac echocardiogram probe. Intravascular ultrasound rotation tracking can also be used for catheter tracking. Moreover, intravascular ultrasound rotation tracking can be used to merge intravascular ultrasound imagery to ultrasound imagery from an additional probe (transthoracic echocardiogram probe, transesophageal echocardiogram probe or intracardiac echocardiogram (ICE) probe). The methods described herein can also be applied in cardiovascular or peripheral vascular applications.

Although intravascular ultrasound rotation tracking has been described with reference to several exemplary embodiments, it is understood that the words that have been used are words of description and illustration, rather than words of limitation. Changes may be made within the purview of the appended claims, as presently stated and as amended, without departing from the scope and spirit of intravascular ultrasound rotation tracking in its aspects. Although intravascular ultrasound rotation tracking has been described with reference to particular means, materials and embodiments, intravascular ultrasound rotation tracking is not intended to be limited to the particulars disclosed; rather intravascular ultrasound rotation tracking extends to all functionally equivalent structures, methods, and uses such as are within the scope of the appended claims.

For example, the external ultrasound imaging probe 156 and the external ultrasound imaging probe 256 have mostly been described as portable or movable probes such as ultrasound probes external to vessel that contains an intravascular ultrasound imaging probe even if still elsewhere within the body of the same human However, beacon signals can also be read and measured by an external sensor such as a fixed sensor or an ultrasound patch on an external surface such as an external surface of the human.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A controller (260) for identifying rotation of an interventional medical device (252), comprising:
a memory (262) that stores instructions; and
a processor (261) that executes the instructions,
wherein, when executed by the processor (261), the instructions cause the controller (260) to execute a process comprising:
receiving a first signal (S410) emitted from the interventional medical device (252) and corresponding to a first predetermined direction relative to the interventional medical device (252);
determining (S420), based on the first signal, a first rotation of the interventional medical device (252) relative to a fixed rotation.

2. The controller (260) of claim 1, wherein the process performed by the controller (260) further comprises:
adjusting (S430) an image taken by the interventional medical device (252) based on the first rotation.

3. The controller (260) of claim 1, wherein the process performed by the controller (260) further comprises:
receiving (S440) a second signal emitted from the interventional medical device (252) and corresponding to a second predetermined direction relative to the interventional medical device (252);
determining (S450), based on the second signal, a second rotation of the interventional medical device (252) relative to the fixed rotation.

4. The controller (260) of claim 3, wherein the process performed by the controller (260) further comprises:
adjusting (S430) a first image taken by the interventional medical device (252) based on the first rotation determined based on the first signal;
adjusting (S460) a second image taken by the interventional medical device (252) based on the second rotation determined based on the second signal, and
aligning (S470) the first image and the second image to construct a 3-dimensional volume.

5. The controller (260) of claim 3,
wherein the first signal and the second signal are emitted at predetermined angles (f1 to f8) from and relative to the interventional medical device (252).

6. The controller (260) of claim 5, wherein at least one of the first rotation and the second rotation is determined based on both the first signal and the second signal.

7. The controller (260) of claim 3, wherein the process performed by the controller (260) further comprises:
adjusting (S460) a second image taken by the interventional medical device (252) based on the second rotation determined based on the second signal, and
aligning (S470) a first image taken by the interventional medical device (252) with the second image to construct a 3-dimensional volume.

8. The controller (260) of claim 1, wherein the first signal is one of a plurality of signals emitted at different frequencies at fixed rotational angles (f1 to f8) relative to the interventional medical device (252).

9. The controller (260) of claim 8, wherein the process performed by the controller (260) further comprises:
weighting (S615), each of a subset of the plurality of signals received at the controller (260); and
determining (S620) the first rotation based on the weighting.

10. The controller (260) of claim 9, wherein the weighting is based on a signal strength of each of the subset of the plurality of signals received at the controller (260).

11. The controller (260) of claim 9, wherein the weighting is based on a power spectrum of the plurality of signals.

12. The controller (260) of claim 1, wherein the interventional medical device (252) comprises an intravascular ultrasound catheter (252).

13. The controller (260) of claim 12, wherein the first signal is emitted while the intravascular ultrasound catheter is inserted into a vessel during a medical intervention.

14. The controller (260) of claim 1, wherein the controller (260) is implemented in a system that includes an ultrasound imaging probe (256), and
wherein imagery from the ultrasound imaging probe (256) is fused with imagery from the interventional medical device (252) based on the first rotation.

15. The controller (260) of claim 1, wherein the controller (260) is implemented in a system that includes an ultrasound imaging probe (256), and
wherein imagery from the ultrasound imaging probe (256) is registered with imagery from the interventional medical device (252) based on the first rotation.

16. The controller (260) of claim 15, wherein the interventional medical device (252) is within a field of view of the ultrasound imaging probe (256) when the first signal is emitted by the interventional medical device (252).

17. The controller (260) of claim 15, wherein the process performed by the controller (260) further comprises:
receiving (S440) a second signal emitted from the interventional medical device (252) and corresponding to a second predetermined direction relative to the interventional medical device (252);
determining (S450), based on the second signal, a second rotation of the interventional medical device (252) relative to the fixed rotation; and
analyzing at least one of a run length of the first signal and a run length of the second signal to enable tracking of the interventional medical device (252) relative to the ultrasound imaging probe (256),
wherein the interventional medical device (252) and the ultrasound imaging probe (256) are correlated to a clock in common.

18. The controller (260) of claim 1, wherein the process performed by the controller (260) further comprises:
fusing, based on determining the first rotation, intravascular ultrasound imagery from the interventional medical device (252) with either transesophageal echocardiogram (TEE) imagery or transthoracic echocardiogram (TTE) imagery.

19. A method for identifying rotation of an interventional medical device (252), comprising:
receiving (S410) a first signal emitted from the interventional medical device (252) and corresponding to a first predetermined direction relative to the interventional medical device (252);
determining (S420), based on the first signal, a first rotation of the interventional medical device (252) relative to a fixed rotation.

20. A system for identifying rotation of an interventional medical device (252), comprising:
the interventional medical device (252);
an ultrasound imaging probe (256) that captures imagery in a space that includes the interventional medical device (252); and
a controller (260) with a memory (262) that stores instructions and a processor (261) that executes the instructions,
wherein, when executed by the processor (261), the instructions cause the controller (260) to execute a process comprising:
receiving (S410) a first signal emitted from the interventional medical device (252) and corresponding to a first predetermined direction relative to the interventional medical device (252);
determining (S420), based on the first signal, a first rotation of the interventional medical device (252) relative to a fixed rotation.
